(19) United States
(12) Patent Application Publication
(10) Pub. No.: US 2025/0213458 A1
(43) Pub. Date: Jul. 3, 2025

(54) Post-Waxing Skin Inflammation Alleviation Kit

(71) Applicant: Addlin Tann, Houston, TX (US)

(72) Inventor: Addlin Tann, Houston, TX (US)

(21) Appl. No.: 18/731,552

(22) Filed: Jun. 3, 2024

Related U.S. Application Data
(60) Provisional application No. 63/616,909, filed on Jan. 2, 2024.

(51) Int. Cl. A61K 8/9789; A61F 7/02; A61K 8/02; A61K 8/34; A61K 8/49; A61K 8/67; A61K 8/9794; A61Q 19/00

(52) U.S. Cl. CPC A61K 8/9789; A61F 7/02; A61K 8/0204; A61K 8/34; A61K 8/4946; A61K 8/676; A61K 8/9794; A61Q 19/005; A61F 2007/0207; A61F 2007/0219; A61F 2007/0226; A61F 2007/0263; A61F 2007/0282; A61K 2800/244; A61K 2800/87

(57) ABSTRACT

A skin inflammation prevention skincare kit designed for use after waxing is disclosed. The kit includes a chillable gel pack containing natural and organic ingredients including aloe vera gel extract, chamomile extract, green tea extract, cucumber extract, menthol, Vitamin C extract, and allantoin. The kit further includes an adhesive applicator that conforms to the body, enabling secure placement of the gel pack on sensitive areas. The kit also includes sanitizing wipes for cleansing the skin before application and Vitamin E wipes for moisturizing and healing after use. The gel pack and gel pad variants are washable, reusable, and eco-friendly. The kit can be tailored for both males and females.

### CROSS-REFERENCE TO RELATED APPLICATION

The present application claims priority to, and the benefit of, U.S. Provisional Application No. 63/616,909, which was filed on Jan. 2, 2024, and is incorporated herein by reference in its entirety.

### FIELD OF THE INVENTION

The present invention generally relates to the field of skincare treatments. More specifically, the present invention relates to a novel skincare kit system for preventing skin inflammation in sensitive areas of the body following waxing. The kit includes chillable gel packs, adhesive applicators, sanitizing wipes, and vitamin E wipes. Gel packs can be placed in the refrigerator for chilling prior to application on the body for inflammation alleviation. The kit can be used by both males and females. Accordingly, the present disclosure makes specific reference thereto. Nonetheless, it is to be appreciated that aspects of the present invention are also equally applicable to other like applications, devices, and methods of manufacture.

### BACKGROUND

By way of background, waxing body parts such as arms, legs, and private areas can be followed by pain, inflammation, and rashes. Usually, individuals prefer waxing hair over shaving for removing unwanted hair from sensitive areas such as the armpit and genital areas. Once the waxing is done in private areas, and if no post-waxing skincare routine is followed, then it may often cause inflammation leading to significant discomfort. Sometimes, individuals may be unable to walk properly due to extreme pain and discomfort in private areas.

The skin in intimate areas is more sensitive in comparison to other parts of the body and when waxing is done, the hair is removed from roots from these sensitive areas, that can irritate the skin and cause inflammation. Inflammation is often followed with redness, swelling, and pain in such areas that may need medical attention if no post-waxing skincare routine is followed. Additionally, waxing may sometimes cause bacterial infection in situations where improper hygiene practices are followed while removing hair from intimate areas. In order to minimize inflammation after waxing in private areas, it's essential to follow proper pre- and post-waxing care routines.

Individuals usually use skin formulations such as soothing moisturizers, oils, topical medicines, and the like on body parts such as hands and legs post-waxing, to get relief from inflammation and pain. However, there are limited ways to alleviate aches and pains in sensitive areas like armpits and genital areas post-waxing hair from these areas. Further, the post-waxing skincare solutions such as moisturizers, topical medicines, or the like used by individuals in the state of the art are not quickly accessible and may take a lot of time to locate skincare formulations and use it at the affected sensitive area, thereby causing a lot of pain and inflammation during that period of time. Furthermore, when in pain due to waxing of sensitive body areas, individuals may be confused and may be unable to choose a particular skincare formulation or medicine to use post-waxing for relieving symptoms of inflammation and swelling. Apart from the above-mentioned problems, males and females may have different skincare routine requirements to accommodate their body contours once the waxing in private areas is done. The post-waxing skin care formulations available in the market do not consider body contour of men and women and fail to fulfill requirements of specific users.

Therefore, there exists a long-felt need in the art for a post-waxing skincare kit that is designed to prevent inflammation in sensitive areas such as genitals and armpits once the waxing is performed to remove hair from these intimate areas. There is also a long-felt need in the art for a waxing skincare kit that prevents pain and swelling caused due to removal of hair from intimate areas of both men and women. Additionally, there is a long-felt need in the art for a post-waxing skincare kit that ensures the sensitive areas are cleaned properly once the waxing of genitals or armpits is done and prevents the users from developing bacterial infections in the waxed body part areas. Moreover, there is a long-felt need in the art for a post-waxing skincare kit that accommodates the body contour requirements of both men and women and fulfills their requirements of a soothing skincare routine post-waxing. Further, there is a long-felt need in the art for a post-waxing skincare kit that ensures safe waxing of sensitive areas such as genitals and armpits of men and women and ensures the users do not require any medical attention to prevent pain due to waxing. Furthermore, there is a long-felt need in the art for a soothing skincare kit that is easily and quickly accessible to the users. Finally, there is a long-felt need in the art for a waxing skincare kit that is easy to use, quickly accessible, relieves pain and swelling, and alleviates any inflammation due to waxing sensitive areas.

The subject matter disclosed and claimed herein, in one embodiment thereof, comprises a skin inflammation prevention skincare kit for use after waxing. The kit includes at least one chillable gel pack configured to be chilled and applied to sensitive areas such as the genitals and armpits after waxing. The chillable gel pack contains natural and organic ingredients including aloe vera gel extract, chamomile extract, green tea extract, cucumber extract, menthol, Vitamin C extract, and allantoin. At least one adhesive applicator is included which has an adhesive lining on a rear surface to adhere to the sensitive area of a human body and configured to conform to the contour of the body, securing (i.e., affixing) the chillable gel pack in place. The kit also includes a pack of individual sanitizing wipes configured to cleanse the skin before applying the chillable gel pack and a pack of Vitamin E wipes configured to moisturize and promote healing after the chillable gel pack is removed.

In this manner, the skin inflammation alleviation skincare kit of the present invention accomplishes all of the foregoing objectives and provides users with a novel skincare kit system for post-waxing care. The gel packs provide immediate cooling relief, which helps to reduce inflammation, redness, and swelling after waxing. The cooling effect of the gel packs helps to numb the skin, providing effective relief from post-waxing pain or discomfort. The adhesive applicators conform to the contours of the body, ensuring that the gel packs stay securely in place on the intended areas such as the armpits, genital regions, or other sensitive parts. The kit can be tailored for both male and female use, with components designed to fit the unique contours of each body type, making it versatile and accommodating.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a skin inflammation prevention skincare kit for use after waxing. The kit includes at least one chillable gel pack configured to be cooled and applied to sensitive areas such as the genitals and armpits. The chillable gel pack contains aloe vera gel extract, chamomile extract, green tea extract, cucumber extract, menthol, Vitamin C extract, and allantoin. At least one adhesive applicator is included and has an adhesive lining on a rear surface to adhere to the surface of a human body and configured to conform to the contour of the body, securing (i.e., affixing) the chillable gel pack in place. The kit also includes a pack of individual sanitizing wipes configured to cleanse the skin before applying the chillable gel pack and a pack of Vitamin E wipes configured to moisturize and promote healing after the chillable gel pack is removed.

In yet another embodiment, a skin inflammation prevention skincare kit for use by females after waxing is disclosed. The kit includes at least one cooling gel pad containing aloe vera gel extract, chamomile extract, green tea extract, cucumber extract, menthol, Vitamin C extract, and allantoin, and configured to provide cooling to sensitive areas after waxing. An adhesive applicator designed to conform to the anatomy of a female body and secure the cooling gel pad in place. A pack of individual sanitizing wipes configured to cleanse the skin before applying the cooling gel pad and a pack of Vitamin E wipes configured to moisturize and promote healing after the cooling gel pad is removed.

In another embodiment, the chillable gel pack includes a gel formulation containing aloe vera gel extract, chamomile extract, green tea extract, cucumber extract, menthol, Vitamin C extract, and allantoin, an outer covering that is transparent, eco-friendly, and skin friendly, and a freezing agent configured to freeze the gel pack in a few minutes.

In another aspect, a method for preventing skin inflammation in sensitive areas after waxing using a skin inflammation prevention skincare kit is described. The method includes the steps of cleansing the skin using a sanitizing wipe, cooling a chillable gel pack or a cooling gel pad from the skincare kit in a refrigerator or freezer, applying the chilled gel pack or gel pad to the skin using an adhesive applicator wherein the pad is positioned between the skin and the applicator, removing the chilled gel pack or gel pad after a period of time, and moisturizing the skin using a Vitamin E wipe from the skincare kit.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the skin inflammation prevention skincare kit of the present invention in accordance with the disclosed structure;

FIG. 2 illustrates perspective views of the cooling gel pad used in the skincare kit of different embodiments of the present invention in accordance with one embodiment of the disclosure;

FIG. 3 illustrates a perspective view of another embodiment of the skin inflammation alleviation skincare kit of the present invention in accordance with the disclosed structure;

FIG. 4 illustrates a perspective view showing the removal of the adhesive protection layer of the adhesive applicator in accordance with the disclosed structure;

FIG. 5 illustrates a perspective view showing refrigeration or freezing of the cooling gel pads of the present invention prior to use in accordance with the disclosed structure; and

FIG. 6 illustrates a side perspective view of a user using an exemplary applicator for securing (i.e., affixing) a cooling gel pack to the armpit in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a post-waxing skincare kit that is designed to prevent inflammation in sensitive areas such as genitals and armpits once the waxing is performed to remove hair from these intimate areas. There is also a long-felt need in the art for a waxing skincare kit that prevents pain and swelling caused due to removal of hair from intimate areas of both men and women. Additionally, there is a long-felt need in the art for a post-waxing skincare kit that ensures the sensitive areas are cleaned properly once the waxing of genitals or armpits is done and prevents the users from catching any bacterial infection in the waxed body part areas. Moreover, there is a long-felt need in the art for a post-waxing skincare kit that accommodates the body contour requirements of both men and women and fulfills their requirements of soothing skincare routine post-waxing. Further, there is a long-felt need in the art for a post-waxing skincare kit that ensures safe waxing of sensitive areas such as genitals and armpits of men and women and ensures the users do not require any medical attention to prevent pain due to waxing. Furthermore, there is a long-felt need in the art for a soothing skincare kit that is easily and quickly accessible to the users. Finally, there is a long-felt need in the art for a waxing skincare kit that is easy to use, quickly accessible, relieves pain and swelling, and alleviates any inflammation due to waxing sensitive areas.

The present invention, in one exemplary embodiment, is a method for preventing skin inflammation in sensitive areas after waxing using a skin inflammation prevention skincare kit. The method includes the steps of cleansing the skin using a sanitizing wipe, cooling a chillable gel pack or a cooling gel pad from the skincare kit in a refrigerator or freezer, applying the chilled gel pack or gel pad to the skin using an adhesive applicator wherein the pad is positioned between the skin and the applicator, removing the chilled gel pack or gel pad after a period of time, and moisturizing the skin using a Vitamin E wipe from the skincare kit.

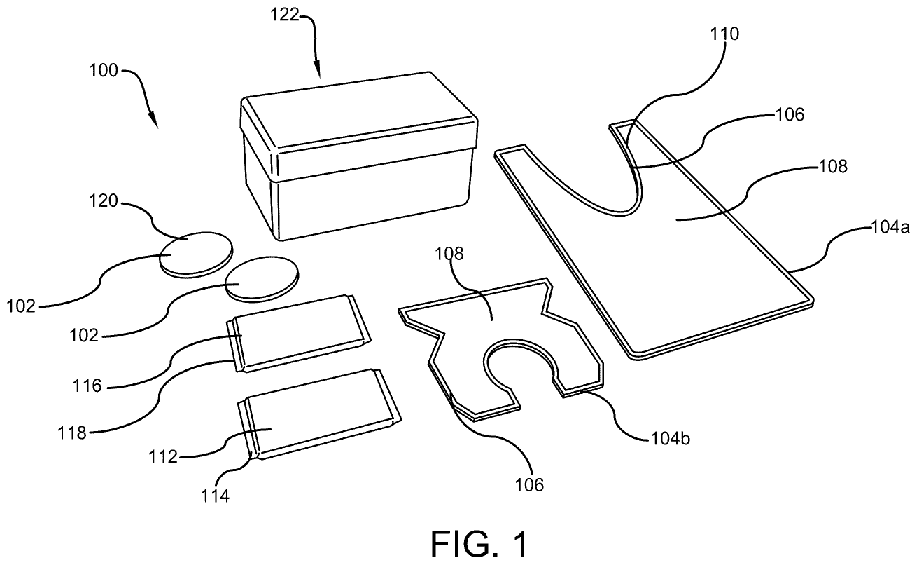

Referring initially to the drawings, FIG. 1 illustrates a perspective view of one potential embodiment of the skin inflammation prevention skincare kit of the present invention in accordance with the disclosed structure. The present embodiment of the skin inflammation prevention skincare kit 100 is configured to be used by males. The skincare kit 100 is designed to alleviate skin inflammation in sensitive areas such as genitals and armpits after waxing. More specifically, the male skincare kit 100 includes at least one chillable gel pack 102 which is configured to be cooled in the refrigerator or freezer for freezing and is designed to be applied directly to the skin after waxing to reduce inflammation, redness, and swelling. The chillable gel pack 102 provides the cooling effect which also helps numb the area on which the chillable gel pack 102 is applied, thereby providing relief from any pain or discomfort. The chillable gel pack 102 can be of any geometric shape for enabling a stable positioning to the target body area.

The skincare kit 100 includes at least one adhesive applicator 104a, 104b. The applicator 104a is designed for covering the armpit and the applicator 104b is designed for use on genital areas of a male. Each adhesive applicator includes an adhesive 106 on the rear surface 108 thereof to adhere to the surface of a human body. The adhesive 106 is skin friendly and includes a pressure adhesive layer 110. The adhesive applicator 104 conforms to contours of the body and enables the chillable gel pack 102 to be securely placed on the skin of a user.

The skin inflammation alleviation skincare kit 100 includes a pack 112 of individual sanitizing wipes 114 wherein a sanitizing wipe is used for cleansing the skin before applying the chillable gel pack 102 and the adhesive applicator 104. The sanitizing wipes 114 help prevent infection by removing bacteria and other contaminants from the waxed area. The skincare kit 100 also includes a pack 116 of Vitamin E wipes 118 wherein a Vitamin E wipe can be used after removing the chillable gel pack 102 for providing moisture and promoting healing, soothing the skin, and aiding recovery.

The chillable gel pack 102 includes 20%-30% w/w aloe vera gel extract, 15%-25% w/w chamomile extract, 15%-25% w/w green tea extract, 15%-25% w/w cucumber extract, 1%-10% w/w menthol, 1%-10% w/w Vitamin C extract, and 1%-10% w/w allantoin. Additionally, the gel pack 102 includes a freezing agent which can freeze the gel pack 102 in a few minutes. Aloe vera gel extract functions as a soothing agent and provides cooling relief and promotes skin healing. Chamomile extract is anti-inflammatory and calming and provides healing for sensitive skin post-waxing. Green tea extract is antioxidant-rich and helps soothe and protect the skin from damage caused due to waxing. Cucumber extract cools and refreshes the skin and helps in reducing swelling and irritation on the waxed skin. Menthol provides a cooling sensation and helps alleviate discomfort on the skin post-waxing. Allantoin soothes and protects the skin, promoting healing and reducing irritation caused due to waxing. Vitamin C is an antioxidant and promotes skin healing and helps brighten the waxed skin.

The chillable gel pack 102 is washable for re-use and can remain frozen for up to 30 minutes at room temperature. The outer covering 120 of the chillable gel pack 102 is transparent, eco-friendly, skin friendly, and does not cause irritation and promotes cooling of the skin to which the chillable gel pack 102 is in direct contact. The kit 100 can come in the form of a box 122 for storing and securing all the components of the kit 100.

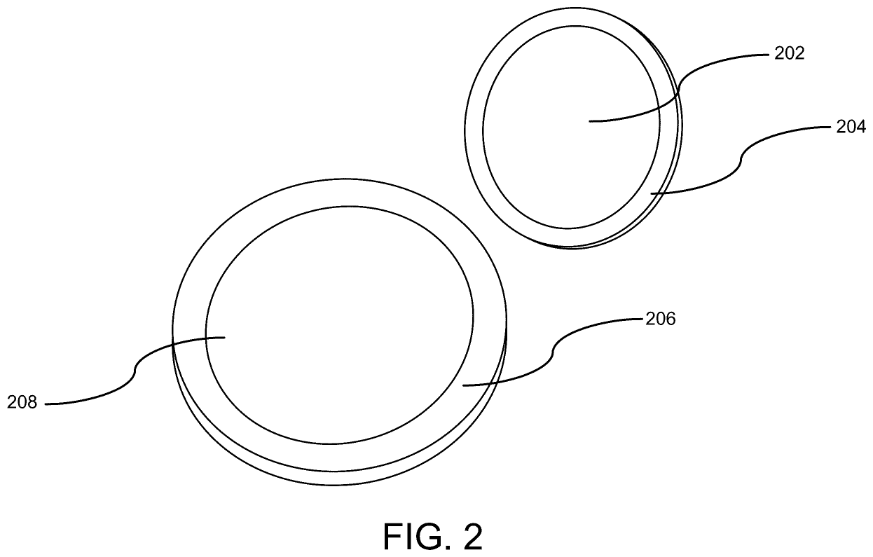

FIG. 2 illustrates perspective views of the cooling gel pad used in the skincare kit of different embodiments of the present invention in accordance with one embodiment of the disclosure. The rear surface 202 of the cooling pad 102 includes an adhesive 204 that can be removably attached to the rear surface 108 of the adhesive applicator 104. The adhesive 204 can depend on the size and shape of the gel pad 102. The front surface 206 can have a logo or indicia 208 disposed thereon for marketing and branding purposes.

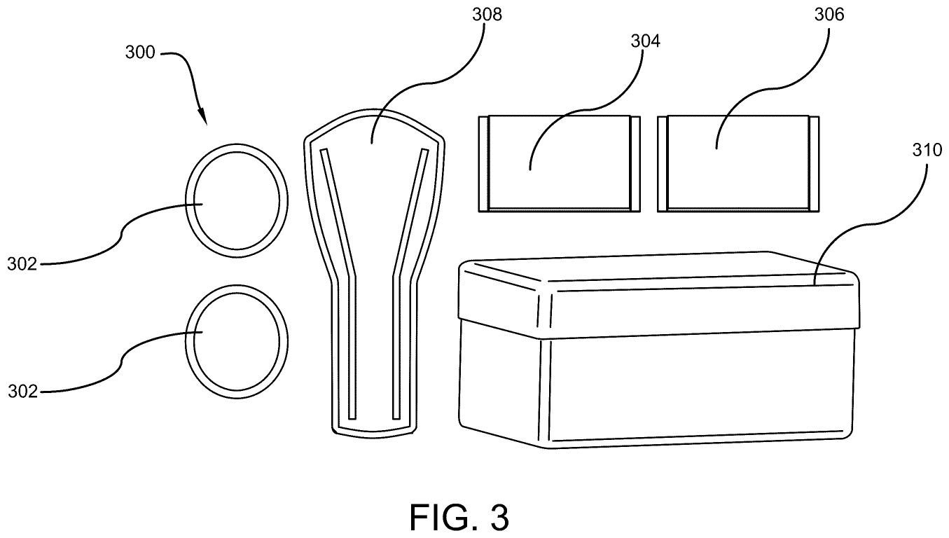

FIG. 3 illustrates a perspective view of another embodiment of the skin inflammation alleviation skincare kit of the present invention in accordance with the disclosed structure. The present embodiment of the skincare kit 300 is designed for use on the female body. More specifically, the skincare kit 300 includes at least one cooling gel pad 302 for providing cooling to the body after waxing. The kit 300 also includes a sanitary wipes pack 304 and a Vitamin E wipes pack 306.

An adhesive applicator 308 designed for application on the genital area of a female is provided in the kit 300. The applicator 308 conforms to the anatomy of the female body and enables secure positioning of the cooling gel pad 302 for providing cooling after waxing. The chillable cooling gel pad 302 includes 10%-20% w/w aloe vera gel extract, 5%-15% w/w chamomile extract, 15%-25% w/w green tea extract, 25%-35% w/w cucumber extract, 10%-20% w/w menthol, 1%-10% w/w Vitamin C extract, and 1%-10% w/w allantoin. Additionally, the gel pack 302 includes a freezing agent and thickening agent to freeze the gel pack 102 in a few minutes inside a freezer. The box 310 can store all the components of the kit 300 securely and easily.

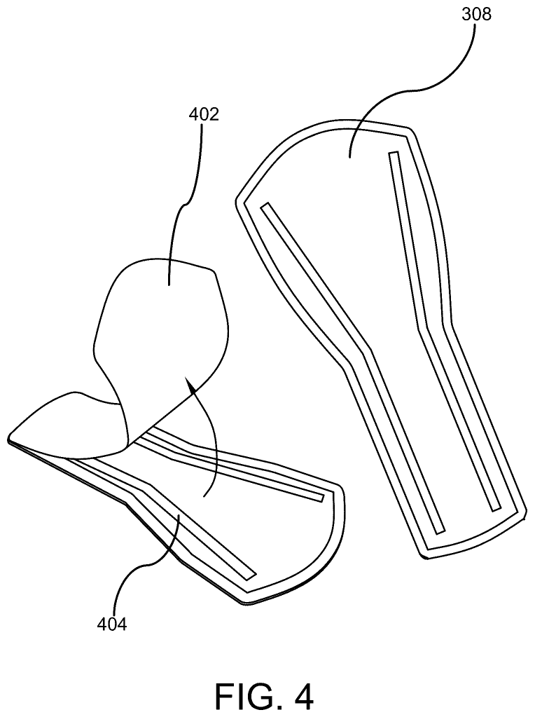

FIG. 4 illustrates a perspective view showing the removal of the adhesive protection layer or lining of the adhesive applicator in accordance with the disclosed structure. The adhesive of each adhesive applicator of the present invention is protected by a corresponding adhesive protection layer or lining 402. As illustrated, the adhesive protection layer or lining 402 protects the adhesive 404 of the applicator 308 and can be peeled off to reveal the adhesive 404 for usage to adhere (i.e., affix) and conform to the body of a user.

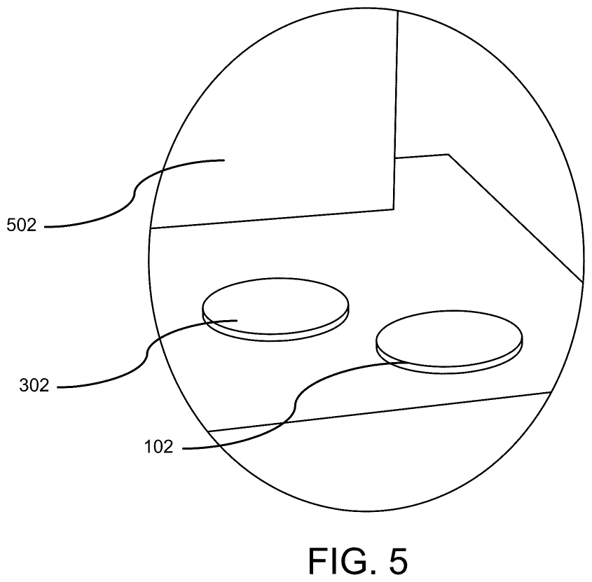

FIG. 5 illustrates a perspective view showing refrigeration or freezing of the cooling gel pads of the present invention prior to use in accordance with the disclosed structure. The cooling gel pads such as cooling gel pads 302, 102 of different embodiments of the present invention can be kept in a freezing device 502 for freezing. The frozen gel pads are used along with applicators for providing soothing and relief from waxing.

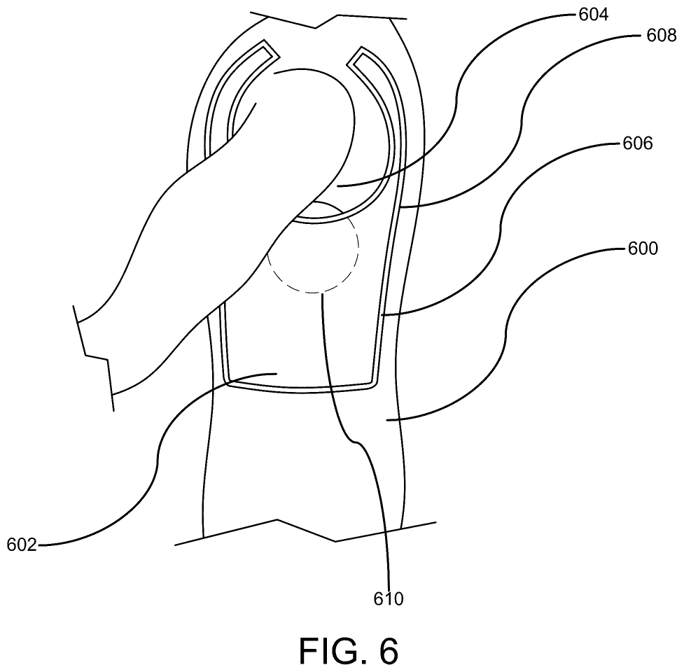

FIG. 6 illustrates a side perspective view of a user using an exemplary applicator for securing (i.e., affixing) a cooling gel pack to the armpit in accordance with the disclosed structure. As illustrated, the applicator 602 (similar to applicator 104a) is applied to the armpit 604 of a user 600 and is secured using the adhesive 606 along the periphery 608 of the applicator 602. A cooling gel pad 610 is secured to the applicator 602 and is placed in the armpit 604 such that the cooling gel pad 610 is positioned between the skin of the user and the applicator 602. The applicator 602 also functions as an absorbent for absorbing any condensation from the cooling gel pad 610, thereby preventing the clothing of a user from getting wet.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “skin inflammation prevention skincare kit”, “male skincare kit”, “skincare kit”, and “skincare kit system” are interchangeable and refer to the skin inflammation alleviation skincare kit 100, 300 of the present invention.

Notwithstanding the foregoing, the skin inflammation alleviation skincare kit 100, 300 of the present invention can be of any suitable size and configuration as is known in the art without affecting the overall concept of the invention, provided that it accomplishes the above-stated objectives. One of ordinary skill in the art will appreciate that the skin inflammation alleviation skincare kit 100, 300 as shown in the FIGS. are for illustrative purposes only, and that many other sizes and shapes of the skin inflammation alleviation skincare kit 100, 300 are well within the scope of the present disclosure. Although the dimensions of the skin inflammation alleviation skincare kit 100, 300 are important design parameters for user convenience, the skin inflammation alleviation skincare kit 100, 300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A skincare kit for alleviating skin inflammation comprising:
a chillable gel pack; and
an adhesive applicator having an adhesive lining and a pressure adhesive layer; and
further wherein said chillable gel pack including 20%-30% w/w aloe vera gel extract, 15%-25% w/w chamomile extract, 15%-25% w/w green tea extract, 15%-25% w/w cucumber extract, 1%-10% w/w menthol, 1%-10% w/w Vitamin C extract, and 1%-10% w/w allantoin.

2. A skincare kit for alleviating skin inflammation of claim 1 further comprising a plurality of sanitizing wipes.

3. A skincare kit for alleviating skin inflammation of claim 2 further comprising a plurality of Vitamin E wipes.

4. A skincare kit for alleviating skin inflammation of claim 1, wherein said chillable gel pack having a freezing agent for freezing said chillable gel pack.

5. A skincare kit for alleviating skin inflammation of claim 1, wherein said chillable gel pack having an outer transparent covering layer.

6. A skincare kit for alleviating skin inflammation of claim 5, wherein said chillable gel pack having an adhesive layer on a rear surface.

7. A skincare kit for alleviating skin inflammation comprising:
a chillable gel pack; and
an adhesive applicator having an adhesive lining and a pressure adhesive layer; and
further wherein said chillable gel pack including 10%-20% w/w aloe vera gel extract,
5%-15% w/w chamomile extract, 15%-25% w/w green tea extract, 25%-35% w/w cucumber extract, 10%-20% w/w menthol, 1%-10% w/w Vitamin C extract, and 1%-10% w/w allantoin.

8. A skincare kit for alleviating skin inflammation of claim 7 further comprising a plurality of sanitizing wipes.

9. A skincare kit for alleviating skin inflammation of claim 8 further comprising a plurality of Vitamin E wipes.

10. A skincare kit for alleviating skin inflammation of claim 9, wherein said chillable gel pack having a freezing agent for freezing said chillable gel pack.

11. A skincare kit for alleviating skin inflammation of claim 10, wherein said chillable gel pack having an outer transparent covering layer.

12. A skincare kit for alleviating skin inflammation of claim 11, wherein said chillable gel pack having an adhesive layer on a rear surface.

13. A method of alleviating skin inflammation, the method comprising the steps of:
providing a chillable gel pack and an adhesive applicator, wherein said adhesive applicator having an adhesive lining and a pressure adhesive layer;
chilling said chillable gel pack;
removing said adhesive lining from said adhesive applicator;
exposing said pressure adhesive layer of said adhesive applicator;
affixing said adhesive applicator to the skin of a user; and
applying said chillable gel pack to said adhesive applicator.

14. The method of alleviating skin inflammation of claim 13, wherein said chillable gel pack including 20%-30% w/w aloe vera gel extract, 15%-25% w/w chamomile extract, 15%-25% w/w green tea extract, 15%-25% w/w cucumber extract, 1%-10% w/w menthol, 1%-10% w/w Vitamin C extract, and 1%-10% w/w allantoin.

15. The method of alleviating skin inflammation of claim 13, wherein said chillable gel pack including 10%-20% w/w aloe vera gel extract, 5%-15% w/w chamomile extract, 15%-25% w/w green tea extract, 25%-35% w/w cucumber extract, 10%-20% w/w menthol, 1%-10% w/w Vitamin C extract, and 1%-10% w/w allantoin.

16. The method of alleviating skin inflammation of claim 13 further comprising the step of freezing said chillable gel pack.

17. The method of alleviating skin inflammation of claim 13 further comprising the step of wiping the skin of the user with a sanitizing wipe prior to affixing said adhesive applicator to the skin of a user.

18. The method of alleviating skin inflammation of claim 17 further comprising the steps of removing said adhesive applicator from the skin of the user and wiping the skin of the user with a Vitamin E wipe.

19. The method of alleviating skin inflammation of claim 16, wherein said chillable gel pack having a freezing agent for freezing said chillable gel pack.

20. The method of alleviating skin inflammation of claim 13, wherein said chillable gel pack having an adhesive layer on a rear surface.
